(19) United States
(12) Patent Application Publication
(10) Pub. No.: US 2011/0045466 A1
(43) Pub. Date: Feb. 24, 2011

(54) Field-effect transistor type biosensor and bio-signal amplification method thereof

(75) Inventors: Ming-Yu Lin, Hsinchu (TW); Yuh-Shyong Yang, Hsinchu (TW); Hsin Chen, Hsinchu (TW)

Correspondence Address: WPAT, PC;INTELLECTUAL PROPERTY ATTORNEYS, 2030 MAIN STREET, SUITE 1300, IRVINE, CA, 92614, US

(73) Assignee: NATIONAL APPLIED RESEARCH LABORATORIES, Taipei (TW)

(21) Appl. No.: 12/572,555

(22) Filed: Oct. 2, 2009

(30) Foreign Application Priority Data
Aug. 24, 2009 (TW) 098128433

(51) Int. Cl. C12Q 1/68; C12M 1/34

(52) U.S. Cl. 435/6; 435/287.2

(57) ABSTRACT

The present invention discloses a field-effect transistor (FET) type biosensor and a bio-signal amplification method. The biosensor comprises a field-effect transistor chip, a biomolecular immobilization layer and at least one primer. The biomolecular immobilization layer is formed on a gate surface of the FET chip or a surface of an external device connected to a gate. The primer used for performing a nucleic acid amplification is immobilized onto the gate surface or the external device surface by binding with the biomolecular immobilization layer, such that an analyte can have a nucleic acid amplification reaction with the primer at room temperature or a constant temperature environment. With an extension of a nucleic acid sequence, the inducing electricity of the FET gate surface can be increased so as to amplify an inspection signal, thereby enhancing the sensitivity of the FET type biosensor effectively.

### BACKGROUND OF THE INVENTION

1. Field of the Invention

The present invention relates to a biosensor and a bio-signal amplification method of the biosensor, in particular to a field-effect transistor type biosensor and a bio-signal amplification method thereof that integrate an isothermal nucleic acid amplification technology

2. Description of the Related Art

A biosensor refers to an analytical device capable of measuring a micro composition by using a biosensing element (such as an enzyme or an antibody) to convert a change quantity of a chemical substance (such as a glucose level, plasma concentration, potassium ion concentration or cholesterol level) in a system into an electronic signal or an optical signal, and its application can meet certain expected requirements for important measurements, particularly the measurement of drugs, metabolic substances, or specific proteins in blood such as a cancer index, or the measurement of interactions between special pathogens such as viruses, bacteria and other biomolecules. In addition, the biosensor can be miniaturized into a chip without requiring fluorescence or enzyme calibration, so that such biosensor provides a convenient way for patients to carry the biosensor and make a measurement timely. In a clinical examination, these properties are very desirable for expediting the testing of a sample and providing a quick and accurate test report for a doctor to achieve a medical treatment effect for a patient.

The biosensor comprises two key portions, respectively: a molecular identifying element obtained from bio-body molecules, tissue parts or somatic cells, wherein this element is arranged for receiving or generating signals of the biosensor; and a physical signal converting element which belongs to a hardware device. It is an important subject for biosensor developers to separate, purify or design a biologically active molecule by a biochemical method, and integrate a precise and quick responding physical transducer to form a biosensor reaction system. At present, the development of biosensors has not reached the mature application stage, but many researches have developed a detection method for detecting a tiny change of quantity of substance in a clinical medicine or drug, and it is an important subject of biosensor developers to provide a biosensor with higher stability and sensitivity.

In the development of a specific protein detection method, a laboratory generally uses an enzyme-linked immunosorbent assay (ELISA) to amplify a first antibody by combining with an secondary antibody of a calibrated enzyme, but this method is affected by a spatial obstacle and the limited epitopes of the secondary antibody on the first antibody, and provides a limited amplification factor. On the other hand, a traditional polymerase chain reaction and an immunological analysis method are integrated to amplify a protein detection signal effectively, and thus an immuno-polymerase chain reaction (Immuno-PCR) is introduced. The immuno-polymerase chain reaction integrates the advantages of the PCR nucleic acid amplification and the identification of the antibody specificity to further amplify the detection of protein through the PCR. However, a general PCR reaction requires repeating a rise and a drop of temperature for three times for a precision temperature control system, and such requirement increases the level of difficulty of designing a high-sensitivity biosensor, particularly a biosensor using electrical measurement (such as an electrochemical biosensor or a nanowire field-effect transistor biosensor). If the temperature rises and drops repeatedly, then the stability of the biosensor will be affected significantly.

### SUMMARY OF THE INVENTION

Therefore, it is a primary objective of the present invention to overcome the aforementioned shortcomings of the prior art by providing a field-effect transistor (FET) type biosensor and a bio-signal amplification method thereof that use an isothermal nucleic acid amplification method such as a rolling circle amplification (RCA) technology to amplify a biosignal to enhance the sensitivity of the field-effect transistor type biosensor.

To achieve the foregoing objective, the present invention provides a field-effect transistor type biosensor comprising a field-effect transistor chip, a biomolecular immobilization layer and at least one primer, wherein the field-effect transistor chip comprises at least one source, at least one drain and at least one gate, and the biomolecular immobilization layer is installed onto a gate surface or a surface of an external device connected to the gate, and the selected primer is immobilized on the biomolecular immobilization layer. In addition, the primer is able to perform an isothermal nucleic acid amplification reaction. The extension of a nucleic acid sequence can be used for directly or indirectly inducing and enhancing the electricity of sensing a field-effect transistor gate surface, such that the inspection signal can be amplified to improve the sensitivity of the field-effect transistor type biosensor effectively.

To achieve the foregoing objective, the present invention provides a bio-signal amplification method for a field-effect transistor type biosensor, comprising the following steps. A field-effect transistor chip with at least one source, at least one drain and at least one gate is provided and a biomolecular immobilization layer is formed on at least one gate surface or a surface of an external device connected to the gate. Then, at least one primer is bound with the biomolecular immobilization layer and immobilized on the gate surface of the field-effect transistor chip or the surface of the external device so as to perform an isothermal nucleic acid amplification reaction.

In summary, the field-effect transistor type biosensor and the bio-signal amplification method thereof in accordance with the present invention have the following one or more advantages:

(1) The present invention integrates the isothermal nucleic acid amplification and the field-effect transistor technologies to achieve the effect of amplifying a DNA nucleic acid signal on a field-effect transistor surface by a reaction conducted at room temperature (without requiring a heating system) or a constant temperature (such as 37° C.) to replace the conventional PCR technology that requires a precise control of three temperature cycles, and the isothermal device is more suitable for precision electrical measurements and comes with an easier design than the PCR temperature control device.

(2) Compared with the conventional PCR quantitative method, the present invention needs not to wait for a sufficient quantity of DNA composite before obtaining signals of the analyte by electrophoresis, but the invention can detect seasonably the analyte concentration by using a change of reaction rate at an early stage of the reaction.

(3) The development of the isothermal nucleic acid amplification technology of the present invention provides a better choice of the protein sensor, not only performing a DNA polymerization with a single-stranded DNA connected to an end of an antibody to amplify the DNA signal, but also performing the amplification at room temperature or a constant temperature environment (such as 37° C.) without requiring a precise temperature control, so as to overcome the temperature control issue of the conventional PCR nucleic acid amplification biosensor.

(4) The field-effect transistor type biosensor of the present invention can be a micro-array biosensor having a better signal amplification effect than the conventional protein array and DNA micro-array chip to overcome the challenge of dynamically measuring a biomolecular reaction by a chip.

(5) The technology of binding a protein (such as an antibody or an aptamer) to a DNA in accordance with the present invention can apply the original DNA detection for the detection of proteins, drugs, organic small molecule, etc. The applications also can be extended to the fields of quick diagnostic testing, home healthcare, cancer screening, virus inspection, and blood donation inspection.

(6) The field-effect transistor type biosensor of the present invention requires no enzyme or fluorescence calibration, or parallel installation of DNA onto the FET, or a double-stranded DNA synthesis for an instant inspection and a direct determination. If aptamer molecules are added, the application can be extended to DNA, RNA, protein and other fields.

(7) The method of the present invention is the same as a real-time PCR quantitative method that can provide the sensitivity for early detections of an analyte. In the meantime, the invention has the advantages of requiring no fluorescence calibration or expensive fluorescence spectrometers over the PCR.

### BRIEF DESCRIPTION OF THE DRAWINGS

FIG. 1 is a flow chart of a bio-signal amplification method for a field-effect transistor type biosensor of the present invention;

FIG. 2 is a schematic view showing the layout of a field-effect transistor chip of a field-effect transistor type biosensor in accordance with an embodiment of the present invention;

FIG. 3 is a schematic view showing the manufacturing flow of a post-CMOS process as depicted in FIG. 2;

FIG. 4 is a schematic view showing a field-effect transistor type biosensor in accordance with an embodiment of the present invention;

FIG. 5 is a schematic view showing a field-effect transistor type biosensor executing a DNA sequence amplification in accordance with an embodiment of the present invention;

FIG. 6A is a schematic view showing the layout of a single-stranded DNA nanotemplate formed by an analyte in accordance with an embodiment of the present invention;

FIG. 6B is a schematic view showing a field-effect transistor type biosensor executing a DNA sequence amplification in accordance with an embodiment of the present invention;

FIG. 7 is a graph of current versus voltage before and after a DNA primer is immobilized on a field-effect transistor in accordance with the present invention; and

FIG. 8 is a graph of current versus voltage before and after a single-stranded DNA template performs a RCA reaction on a field-effect transistor in accordance with the present invention.

### DETAILED DESCRIPTION OF THE PREFERRED EMBODIMENTS

The technical characteristics of the present invention will become apparent with the detailed description of the following preferred embodiments and related drawings.

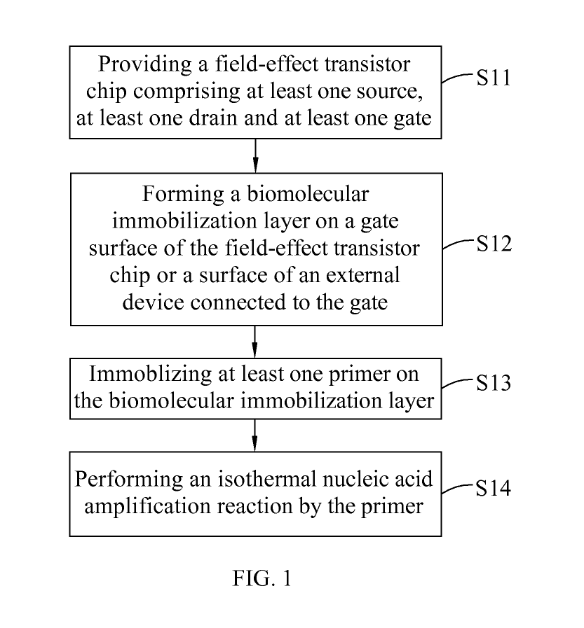

With reference to FIG. 1 for a flow chart of a bio-signal amplification method for a field-effect transistor type biosensor of the present invention, the bio-signal amplification method comprises the following steps. In step S11, a field-effect transistor chip comprising a source, a drain and a gate is provided. In step S12, a biomolecular immobilization layer is formed on a gate surface of the field-effect transistor chip or a surface of an external device connected to the gate. In step S13, at least one primer is immobilized on the biomolecular immobilization layer, and in step S14, an isothermal nucleic acid amplification reaction is performed by the primer. Since the nucleic acid sequence is extended and amplified to enhance the electricity of sensing the field-effect transistor gate surface, so as to amplify the biosignal and improve the sensitivity of the field-effect transistor type biosensor. In addition, the nucleic acid amplification reaction can be held at room temperature or a constant temperature environment, and thus the instability of the measurement caused by a temperature change can be solved.

The field-effect transistor chip can be a nanowire field-effect transistor (NWFET) chip, a carbon nanotube field-effect transistor (CNTFET) chip, an ion-sensitive field-effect transistor (ISFET) chip, an oxide-semiconductor field-effect transistor (OSFET) chip or a field-effect transistor chip fabricated by a complementary metal oxide semiconductor (CMOS) process. In addition, the gate surface or the surface of an external device connected to the gate is made of a material comprising a silicon-based material. The biomolecular immobilization layer is made of a material capable of producing a biomolecular binding, and the isothermal nucleic acid amplification reaction comprises a rolling circle amplification reaction in which a DNA polymerase and a single-stranded circular DNA template formed by an analyte are added onto the biomolecular immobilization layer, such that the at least one primer can perform the rolling circle amplification reaction with the single-stranded circular DNA template by the DNA polymerase. The selected primer comprises a DNA segment, a RNA segment, an aptamer or an antibody, and all of these primers comprise a special sequence that can induce a nucleic acid amplification reaction, and the detectable analyte comprises a DNA sequence, a RNA sequence, a protein, a small molecule, a drug, etc.

The field-effect transistor type biosensor of the present invention can amplify the DNA sequence of an analyte significantly. For example, a 18mer DNA can be duplicated and amplified to a 3000mer DNA, and since the DNA sequence can carry a negative electric charge, the elongated DNA sequence definitely carries more negative charges that can induce the gate surface of the field-effect transistor chip to produce more positive charges, so that the inspection signal will become larger accordingly, and the sensitivity of the field-effect transistor type biosensor can be enhanced effectively.

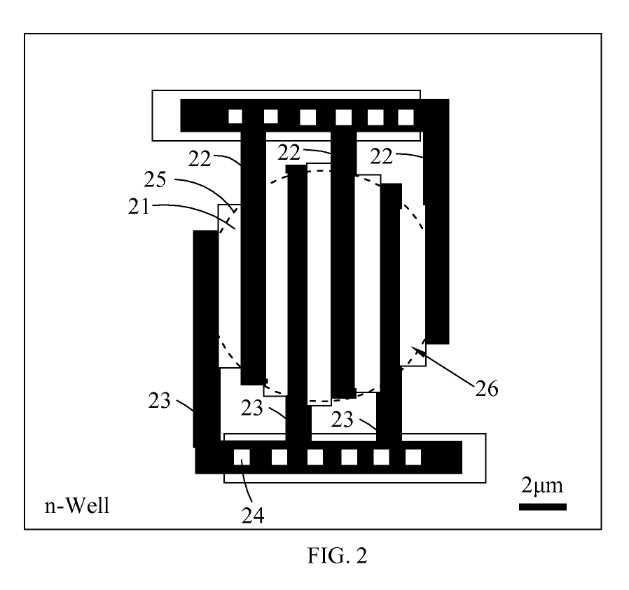

With reference to FIG. 2 for a schematic view showing the layout of a field-effect transistor chip of a field-effect transistor type biosensor in accordance with an embodiment of the present invention, a field-effect transistor chip is fabrication by an n-well complementary metal oxide semiconductor (CMOS) process. In FIG. 2, the field-effect transistor chip comprises a plurality of gates 21, a plurality of drains 22, a plurality of sources 23, and an electrode contact 24 for pulling each electrode, wherein a dashed circle 25 indicates a sensing area 26 of the biosensor, and all materials disposed on a gate-oxide layer in the sensing area 26 are removed to enhance the sensitivity to form an open-gate field-effect transistor structure, and potential changes above the gate-oxide layer are transformed directly into changes in a channel current, which flows between the drain 22 and the source 23. In addition, the field-effect transistor chip structure can executes the maximum transconductance within the specific sensing area 26.

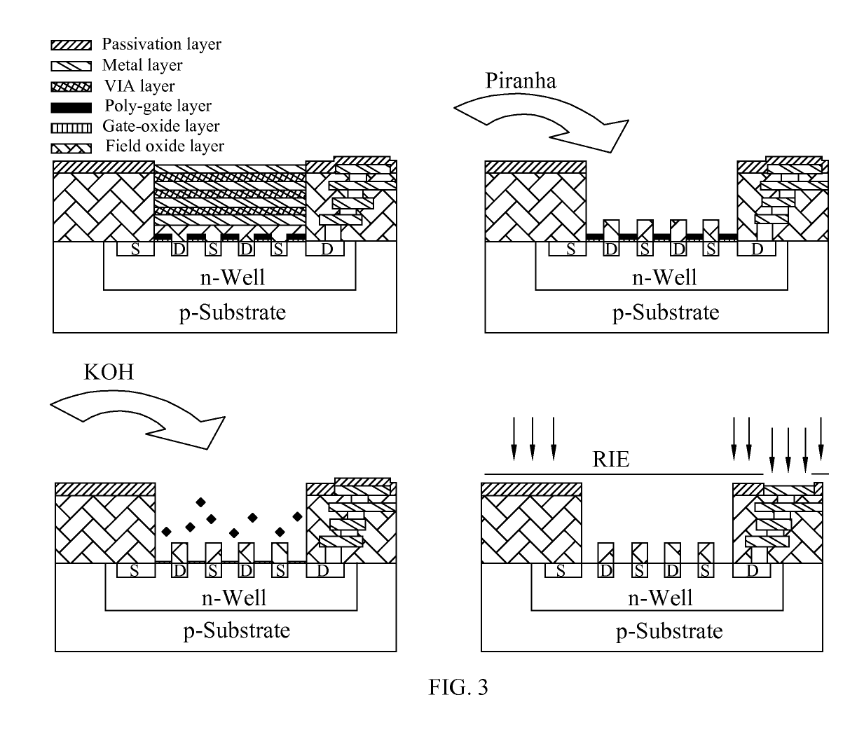

With reference to FIG. 3 for a schematic view showing the manufacturing flow of a post-CMOS process as depicted in FIG. 2, the post-CMOS process is provided for manufacturing a field-effect transistor at the die level. In FIG. 3(A), a standard TSMC 0.35 μm CMOS process is used for manufacturing a p-channel field-effect transistor (p-channel FET), wherein metal layers are provided for defining the sensing area. In FIG. 3(B), the metal layers are removed by wet etching with “piranha” at 85° C., so that the poly-gates of the field-effect transistor are exposed, and the reactive-ion etching (RIE) is applied for five minutes to remove a thin silicide layer above the poly-gates. In FIG. 3(C), a ratio of potassium hydroxide: deionic water equal to 1:2 is used for wet etching the poly-gates at 80° C. for 20 seconds to expose the gate-oxide layer. In FIG. 3(D), with a fraction of a silicon wafer functioning as a shadow mask, a passivation layer disposed over a bonding pad is exposed by the RIE.

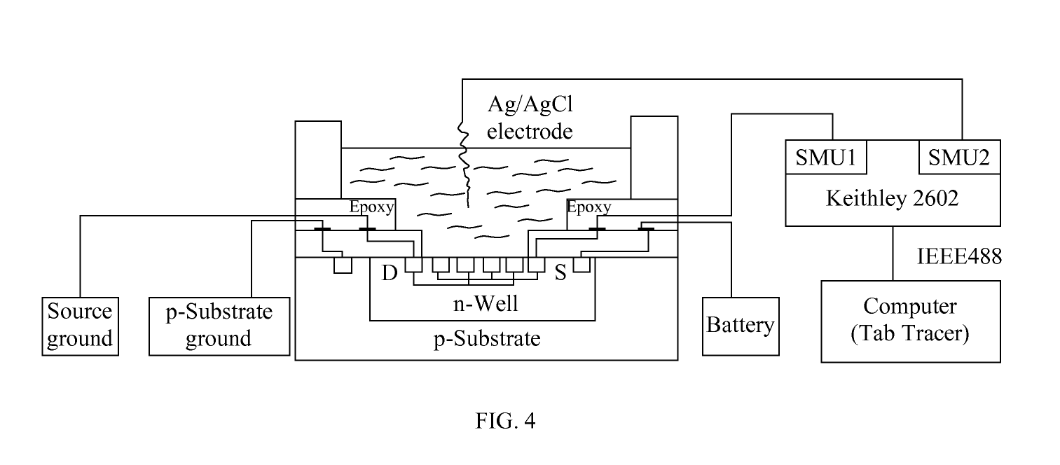

After the post-CMOS process, the manufactured field-effect transistor chip is wire-bonded to a printed circuit board, to which a glass O-ring is attached to form a bath, as shown in FIG. 4. The entire field-effect transistor chip surface except for the field-effect transistor area is coated with industrial epoxy for preventing a short circuit introduced by a solution in the bath. In addition, a Keithley 2602 Series SourceMeter is used to bias and measure the field-effect transistor type biosensor with the source meter configured through the software “Tab Tracer”. As the gate material of the field-effect transistor is substituted by the solution in the glass O-ring, a silver/silver chloride electrode will be employed for providing a DC-bias for the solution. With the voltages of the drain (D) and the solution kept at 0 volt, the channel current of the field-effect transistor is measured when the voltage of the source (S) is swept from 0 volt to 3 volt with a step of 50 millivolts. In addition, the current-voltage relationships at different stages of DNA synthesization can be measured and transmitted to a computer through an IEEE488 cable.

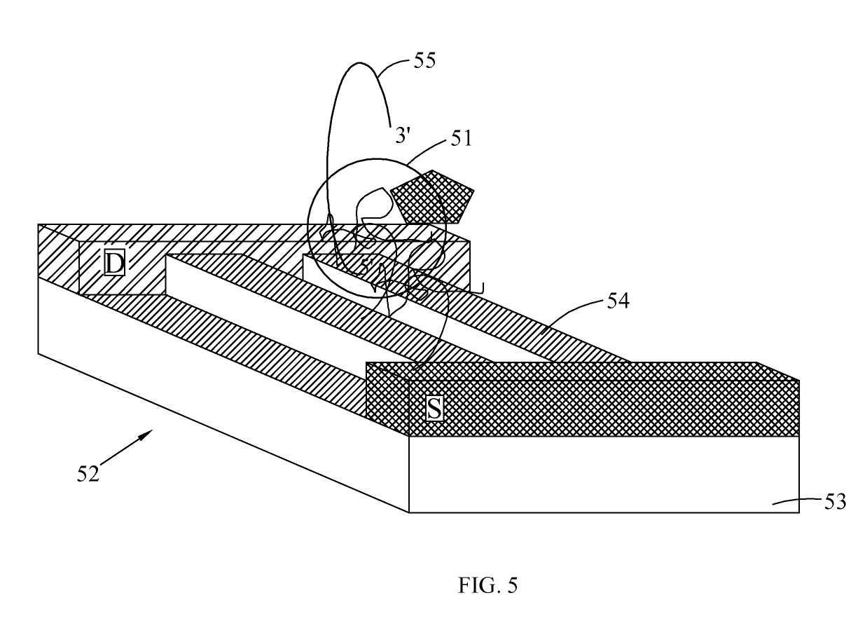

With reference to FIG. 5 for a schematic view showing a field-effect transistor type biosensor executing a DNA sequence amplification in accordance with an embodiment of the present invention, the single-stranded DNA nanotemplate 51 is situated on a surface 54 of a gate 53 of a field-effect transistor 52 of a field-effect transistor chip manufactured according to the manufacturing procedure as depicted in FIG. 3, and is employed to perform an in situ rolling circle amplification reaction with a DNA primer 55 immobilized on a biomolecular immobilization layer (not shown in the figure). The biomolecular immobilization layer is a self-assembly monolayer (SAM) which acts as a covalent linker between the DNA primer 55 and a silicon oxide surface of the gate, so that the analyte can be situated at a closer position to the sensing gate, or even bound directly onto the gate. In this embodiment, the rolling circle amplification (RCA) reaction of the present invention comprises the following three main technologies:

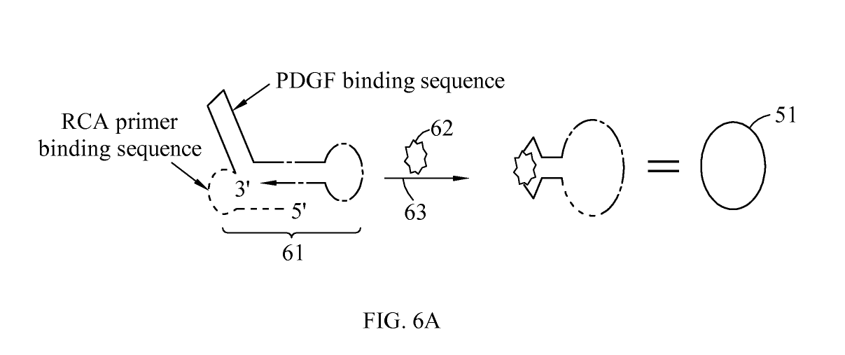

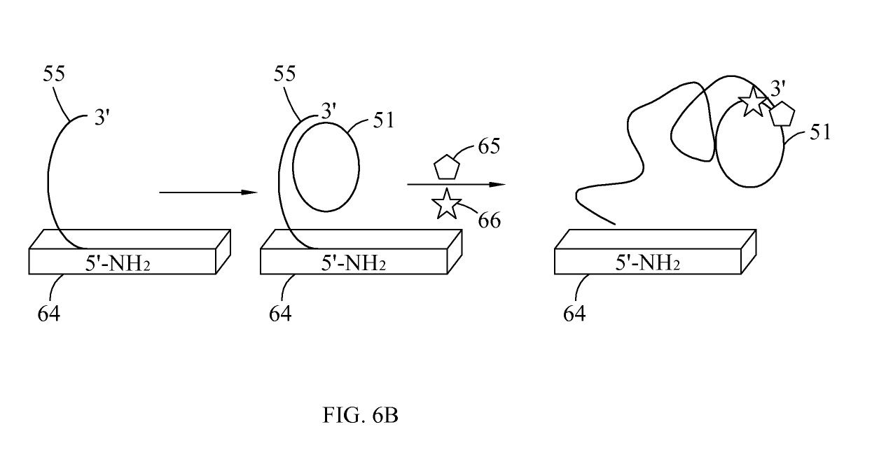

(1) The PDGF aptamer can be induced by a platelet-derived growth factor (PDGF) to cause a conformational switch. In FIG. 6A, the PDGF aptamer 61 can identify a PDGF protein 62, and induce the PDGF aptamer to have a deformation 63 to form a single-stranded circular DNA template 51.

(2) The rolling circle amplification reaction is used for duplicating and amplifying a single-stranded DNA nanotemplate. In FIG. 6B, a circular PDGF aptamer (which is a single-stranded DNA nanotemplate 51) is added into a solution above the field-effect transistor type biosensor as depicted in FIG. 4, and the aptamer is complementary to the DNA primer 55 immobilized on the gate surface 64. A phi29 DNA polymerase 65 and a T4 gene-32 protein 66 are then added to start a rolling circle amplification reaction with the single-stranded DNA nanotemplate 51 and the DNA primer 55, wherein the phi29 DNA polymerase 65 acts both on the DNA polymerization and the single-stranded DNA displacement, and the T4 gene-32 protein 66 acts as a single-stranded DNA binding protein.

(3) After the single-stranded DNA nanotemplate is used for polymerization and amplification, the electricity of the field-effect transistor is induced and increased to improve the sensitivity of the biosensor.

The self-assembly monolayer for this embodiment is formed onto a gate surface of the field-effect transistor by silanization using a 3-aminopropyl triethoxysilane (APTES).

The detailed procedure of the experiment is described as follows:

Firstly, the cover glass substrate of a field-effect transistor is washed by an ethanol solution to remove contaminants, and incubated with a 2.0% APTES alcohol solution for approximately 30 minutes, and heated at 120° C. for 10 minutes to remove any remaining alcohol.

Secondly, the substrate is treated with a solution containing 2.5% glutaraldehyde and 4 mM sodium cyanoborohydride for one and half hours followed by water wash.

Finally, the 500 nM 5′-aminomodified primer is coupled to the glass at 4° C. overnight, and the primer is coupled onto a self-assembly monolayer of the substrate.

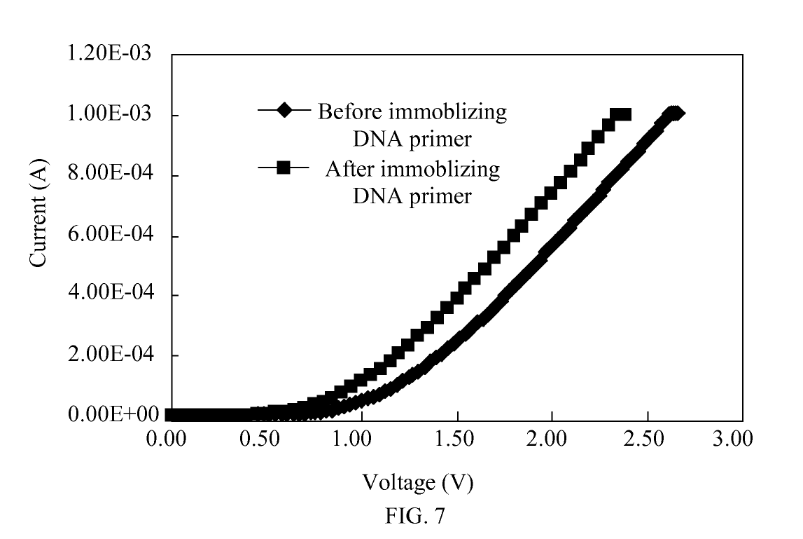

With reference to FIG. 7 for a graph of current versus voltage before and after a DNA primer is immobilized on a self-assembly monolayer of a field-effect transistor chip in accordance with the present invention, it is found that after the DNA primer is immobilized on the self-assembly monolayer, the current-voltage curve shifts to the left of the current-voltage curve before the DNA primer is immobilized. The left shift indicates that more negative charges are accumulated on the surface of the field-effect transistor because the current of a p-channel transistor increased with its voltage. This result agrees with the fact that the DNA primer is negatively-charged, demonstrating the capability of the field-effect transistor biosensor of the present invention to detect the immobilization of the DNA primer.

In addition, the aptamer used in the present invention can be a single-stranded DNA molecule with a three-dimensional structure produced by an artificial synthesis, and having the capability of recognizing a target protein, PDGF, and the aptamer can be transformed into a circular form during the recognition process. The experimental method is described as follows:

5 nM PDGF is incubated with 40 nM PDGF aptamer in a nucleic acid ligation reaction, and then the ligation reaction is terminated by heating at 95° C. for 5 minutes.

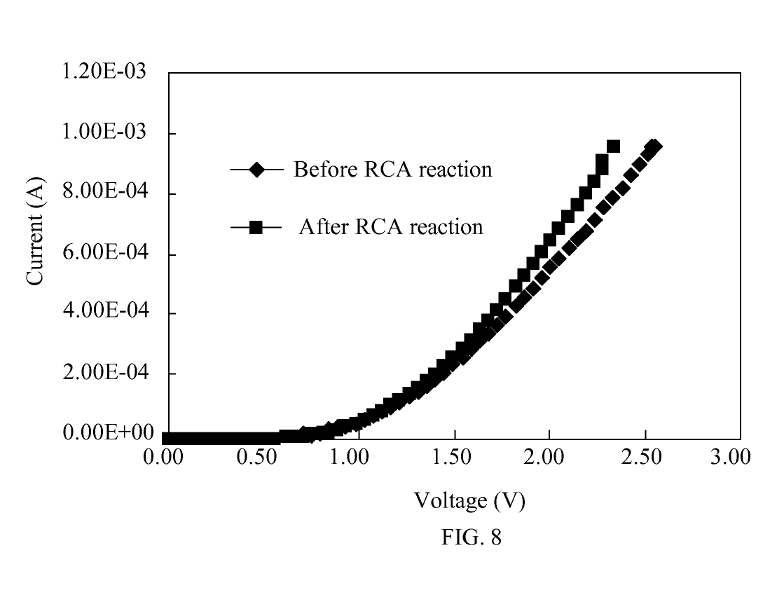

The obtained circular single-stranded DNA can be added into a RCA reaction chamber of the field-effect transistor chip to start the RCA reaction. The current versus voltage graph measured before and after the reaction takes place is shown in FIG. 8. In FIG. 8, it is found that after the single-stranded DNA template has gone through the rolling circle amplification reaction, the obtained current-voltage curve shifts to the left, indicating that the single-stranded DNA template is duplicated and amplified, such that the electricity of sensing the field-effect transistor is enhanced.

In summation of the description above, the field-effect transistor type biosensor of the present invention can achieve the effect of polymerizing and amplifying the single-stranded DNA template formed by the analyte to improve the accuracy of detecting the field-effect transistor type biosensor.

While the invention has been described by means of specific embodiments, numerous modifications and variations could be made thereto by those skilled in the art without departing from the scope and spirit of the invention set forth in the claims.

What is claimed is:

1. A field-effect transistor type biosensor, comprising:
a field-effect transistor chip, comprising at least one source, at least one drain and at least one gate;
a biomolecular immobilization layer, disposed on a surface of the at least one gate or a surface of an external device connected to the gate; and
at least one primer, immoblized on the biomolecular immobilization layer, and performing an isothermal nucleic acid amplification reaction.

2. The field-effect transistor type biosensor of claim 1, wherein the field-effect transistor chip comprises a nanowire field-effect transistor chip, a carbon nanotube field-effect transistor chip, an ion-sensitive field-effect transistor chip, an oxide-semiconductor field-effect transistor chip or a field-effect transistor chip fabricated by a semiconductor process.

3. The field-effect transistor type biosensor of claim 2, wherein the semiconductor process comprises a complementary metal oxide semiconductor (CMOS) process.

4. The field-effect transistor type biosensor of claim 1, wherein the surface of the at least one gate or the surface of the external device connected to the gate is made of a material comprising a silicon-based material.

5. The field-effect transistor type biosensor of claim 4, wherein the biomolecular immobilization layer is a self-assembly monolayer.

6. The field-effect transistor type biosensor of claim 5, wherein the self-assembly monolayer is made of a material comprising 3-aminopropyl triethoxysilane (APTES).

7. The field-effect transistor type biosensor of claim 6, wherein the self-assembly monolayer is formed on the surface of the at least one gate or the surface of the external device by silanization.

8. The field-effect transistor type biosensor of claim 1, wherein the biomolecular immobilization layer comprises a material capable of forming a biomolecular binding.

9. The field-effect transistor type biosensor of claim 1, wherein the isothermal nucleic acid amplification reaction comprises a rolling circle amplification reaction.

10. The field-effect transistor type biosensor of claim 9, wherein a DNA polymerase used for the rolling circle amplification reaction comprises a phi29 DNA polymerase.

11. The field-effect transistor type biosensor of claim 1, wherein the at least one primer comprises a DNA segment, a RNA segment, an aptamer or an antibody.

12. The field-effect transistor type biosensor of claim 1, wherein an analyte used for the isothermal nucleic acid amplification reaction comprises a DNA sequence, a RNA sequence, a protein, a small molecule or a drug.

13. A bio-signal amplification method for a field-effect transistor type biosensor, comprising the steps of:
providing a field-effect transistor chip comprising at least one source, at least one drain and at least one gate;
forming a biomolecular immobilization layer on a surface of the at least one gate or a surface of an external device connected to the gate;
immoblizing at least one primer onto the biomolecular immobilization layer; and
performing an isothermal nucleic acid amplification reaction by the at least one primer.

14. The bio-signal amplification method for a field-effect transistor type biosensor as recited in claim 13, wherein the isothermal nucleic acid amplification reaction comprises a rolling circle amplification reaction in which a DNA polymerase and a single-stranded circular DNA template formed by an analyte are added onto the biomolecular immobilization layer, such that the at least one primer performs the rolling circle amplification reaction with the single-stranded circular DNA template by the DNA polymerase.

15. The bio-signal amplification method for a field-effect transistor type biosensor as recited in claim 14, wherein the DNA polymerase comprises a phi29 DNA polymerase.

16. The bio-signal amplification method for a field-effect transistor type biosensor as recited in claim 13, wherein the field-effect transistor chip comprises a nanowire field-effect transistor chip, a carbon nanotube field-effect transistor chip, an ion-sensitive field-effect transistor chip, an oxide-semiconductor field-effect transistor chip or a field-effect transistor chip fabricated by a semiconductor process.

17. The bio-signal amplification method for a field-effect transistor type biosensor as recited in claim 15, wherein the semiconductor process comprises a complementary metal oxide semiconductor (CMOS) process.

18. The bio-signal amplification method for a field-effect transistor type biosensor as recited in claim 13, wherein the surface of the at least one gate or the surface of the external device connected to the gate is made of a material comprising a silicon-based material.

19. The bio-signal amplification method for a field-effect transistor type biosensor as recited in claim 17, wherein the biomolecular immobilization layer is a self-assembly monolayer.

20. The bio-signal amplification method for a field-effect transistor type biosensor as recited in claim 18, wherein the self-assembly monolayer is made of a material comprising 3-aminopropyl triethoxysilane (APTES).

21. The bio-signal amplification method for a field-effect transistor type biosensor as recited in claim 19, wherein the self-assembly monolayer is formed on the surface of the at least one gate or the surface of the external device by silanization.

22. The bio-signal amplification method for a field-effect transistor type biosensor as recited in claim 13, wherein the at least one primer comprises a DNA segment, a RNA segment, an aptamer or an antibody.

23. The bio-signal amplification method for a field-effect transistor type biosensor as recited in claim 13, wherein an analyte used for the isothermal nucleic acid amplification reaction comprises a DNA sequence, a RNA sequence, a protein, a small molecule or a drug.
